(19) United States
(12) Patent Application Publication
(10) Pub. No.: US 2004/0009096 A1
(43) Pub. Date: Jan. 15, 2004

(54) Substantially inertia free hemodialysis

(76) Inventor: Parris S. Wellman, Hillsborough, NJ (US)

Correspondence Address: Parris S. Wellman, 61-31 Taurus Drive, Hillsborough, NJ, 08844, US

(21) Appl. No.: 10/455,543

(22) Filed: Jun. 5, 2003

Related U.S. Application Data
(60) Provisional application No. 60/387,135, filed on Jun. 8, 2002.

(51) Int. Cl. A61M 37/00; A61M 1/34; C02F 1/44

(52) U.S. Cl. 422/44; 604/5.01; 210/646

(57) ABSTRACT

This invention is a membraneless filtration device and a method for performing hemodialysis on a patient using it and methods of manufacturing the filtration device. In a preferred embodiment, the filter is composed of at least one channel where the dialysate circumferentially surrounds the blood to reduce or eliminate the need for anticoagulation through reduced hemolysis and thrombogenicity. In the preferred embodiment the filter is configured to be used with a typical dialysis machine and hemodialysis is separated into an iso- or hyper-volemic filtration phase, followed by a phase where the patient's blood volume is reduced to normal levels. In another embodiment the device is adapted to be implanted or worn externally to serve as a portable artificial kidney. The device described herein could also find application as a blood oxygenator in cardiopulmonary bypass, or for use as an artificial liver or lung.

### FIELD OF THE INVENTION

This invention is generally related to the field of blood filtration and more particularly to hemodialysis. The invention is particularly useful for providing extracorporeal filtration of blood contaminants in a physiologically compatible manner, for example as a portable artificial kidney or artificial liver.

### BACKGROUND OF THE INVENTION

End stage renal disease, ESRD, afflicts some 977 patients per million in the United States alone. This implies that there are approximately 280,000 patients in the US, and there are an equal number in Europe. The disease has a prevalence of 329 per million, implying that 90,000 new patients are afflicted with ESRD a year. However, given the high mortality rate in the United States (approaching 25%/year) the actual population growth rate is about 9%. There are a number of treatments for ESRD including hemodialysis, peritoneal dialysis and kidney transplantation. Nearly 70% of the patients having ESRD are on chronic, maintenance hemodialysis. The aim of treatment is to replace the function of the kidneys in removing contaminants from the blood and to maintain the appropriate blood volume, whether hemodialysis or peritoneal dialysis is employed.

Hemodialysis (HD) was invented early in the ₂₀th century, with the first successful chronic cases being performed in the 1950s. It is also known as the artificial kidney. It is based on the mechanism of diffusion through a semi-permeable membrane. The blood is brought in contact with one side of the membrane, and a dialysate solution (purified water with various concentrations of potassium, sodium and other agents designed to balance the diffusion from the blood across the membrane) is put on the other side of the membrane. Diffusion drives the solutes through the membrane until equilibrium is reached. Volume control is achieved by controlling the pressure differential across the membrane—osmosis drives water out of the blood and into the dialysate. This type of dialysis is typically performed 3-4 days a week for 3-4 hours each day in the United States. In the United States, approximately 200,000 ESRD patients receive hemodialysis, and this number is growing at an annual rate of approximately 9%.

Hemofiltration (HF): This was more recently invented, and typically accompanies hemodialysis. The new polysulfone dialysis membranes are somewhat porous to the dialysate and this dialysate is entrained by the blood flow. This mixing of clean dialysate and blood has come to be known as hemofiltration. This type of dialysis is accompanies hemodialysis and is said to be responsible for better removal of middle molecules from the blood.

Peritoneal dialysis (PD): In this type of dialysis, the patient's own peritoneum serves as the dialysis membrane. Dialysate is irrigated into the peritoneal cavity and the blood exchange occurs there. PD allows for easy home dialysis and is portable. However, in certain patients, changes in the peritoneal lining occur and the effectiveness of the dialysis diminishes. This technique is also accompanied by a relatively large rate of morbidity because of peritoneal infections. On the other hand, it does allow for home dialysis, but typical dialysis times are quite long (8 hours) and usually need to be done overnight.

Problems with HD:

Of the 280,000 ESRD patients in the United States in 2000, approximately 60,000 of them received kidney transplants and 200,000 were on chronic hemodialysis. The remainder received peritoneal dialysis. While kidney transplantation is the ideal, lack of available kidneys to transplant is the limiting factor. As was stated before, there is considerable concern than PD cannot achieve effective dialysis in many patients because of inadequate perfusion of the peritoneal lining, or because of changes induced in the peritoneal lining because of the dialysis. It is frequently thought that peritoneal dialysis is not appropriate in patients who lack residual renal function. The procedure also has relatively high morbidity with patients frequently treated (an average of 5 times/year, with one hospitilization) for peritontitis. The principal advantage of peritoneal dialysis is its portability—patients can perform home dialysis using a portable machine.

In contrast, while it is possible to provide adequate training to make home hemodialysis possible, this is typically not the case as it requires a much higher level of skill, and frequently a partner to help in the process. Thus, HD remains the province of specialized dialysis clinics that employ specially trained staff. In addition, the costs associated with home hemodialysis can be prohibitively high.

For all forms of dialysis, in the United States, morbidity and mortality remain unacceptably high. Average life expectancy is about 10 years (approximately the same as for cancer patients). Mortality is about 25%/year. Most of these fatalities are due to cardiac events. A likely cause of these events is hypertension as a result of inadequate dialysis. In other countries, notably France, Italy and Japan, there is a much lower mortality and morbidity rate which is likely because they dialyze patients for much longer times or more frequently. There is a growing body of literature to support daily hemodialysis, for shorter durations, to reduce morbidity and mortality.ⁱ It is hypothesized that frequent dialysis leads to better middle molecule removal and better control of total fluid load which in turn leads to better control of blood pressure and a concomitant reduction in morbidity and mortality.

Anemia often accompanies ESRD. This is due to a lack of erythropoetin production in the kidneys. It also seems likely that there is a component of hemolysis (breaking of red blood cells due to shear or other action) involved. Obviously, elimination of hemolysis is a good goal in any case.

Another problem with hemodialysis is the lack of removal of certain proteins (they are unable to pass through the membrane because of their high molecular weight). Additionally, phosphates are typically not removed efficiently. They too have trouble passing through the membrane. Taken together, these adverse effects result in people on HD feeling sick much of the time.

Further, because of the exposure of the blood to non-biological materials, there is a need for the blood to be anticoagulated using heparin or other anticoagulation agent. The dose of heparin must then be reversed (using protamine) before the patient leaves the dialysis clinic. Some patients have difficulty tolerating the doses of heparin and protamine required for effective treatment. Further, it has been recognized that current dialysis membranes work with less side effects for the patient if they are reused. This is likely due to the accumulation of proteins on the surface of the membrane which reduces the incomparability with the blood. This accumulation of proteins may reduce the complement activation that can be associated with hemodialysis, and likely help to prevent system inflammatory response. Unfortunately, these proteins also reduce the efficiency of the dialysis membrane, necessitating longer dialysis times. Further, the risk of infection greatly increases with reuse because it can be difficult to re-sterilize the dialysis membranes.

A further difficulty of these hemofiltration units is the relatively large priming volume required for their use. In some cases, as much as 500 ml of blood is required to effectively prime the circuit and filter. This large amount of blood removed from the system of the patient may not be well tolerated, and can lead to light-headedness and even loss of conciousness. In extreme cases brain ischemia is even possible.

A further problem with blood flow over membranes is that at the contact of the blood and the membrane, a large shear force is created. These shear forces could result in hemolysis (breaking of red blood cells) that could contribute to the chronic anemia that many kidney dialysis patients experience. Hemolysis is managed in current filters by keeping blood to membrane interface velocity low—this in turn increases the amount of time required for dialysis.

This invention is not limited to use in hemodialysis. Similar problems are encountered with blood oxygenators for cardiopulmonary bypass (CPB). Here, instead of removing urea and other contaminants from the blood, the filters are used to introduce oxygen and remove dissolved carbon dioxide. These filters typically have much larger surface area than hemodialysis filters owing to the much larger volumetric flow rates that they need to deal with (on the order of 5 liters/minute versus 100s of milliliters/min for hemodialysis filters). As a result, the blood cells experience greater shear stress which could lead to greater hemolysis. Exposure to the membranes has been implicated in the compliment activation phenomena and the systemic inflammatory response that CPB patients often experience.

### SUMMARY OF THE INVENTION

The principal aim of this invention is to provide a method that makes daily, short duration (1 hour) hemodialysis practical. Decreasing the dialysis time to one hour will allow patients more freedom in their dialysis treatments, and will increase the utilization of clinics and personnel and thereby reduce total costs. A further aim of this invention is to increase the quality of the dialysis by making it practical to perform daily hemodialysis and thereby reduce the mortality rate. This invention provides an alternative to membrane dialysis through the use of shaped flows and substantially inertia free flows (flows at very low Reynolds numbers).

FIG. 1 shows a picture of one possible implementation of the system. Locating the dialysis module, 1, in close proximity to the patient, and making it small in size, reduces priming volumes and reduces the risk of adverse effects for the patient, like hypovolemic shock or hypotension. A standard dialysis machine or other fluid drive mechanism is connected to the dialysate inlet port, 5, and the exit port, 6, while the blood is taken from a synthetic graft or fistula through the blood inlet port, 3, and returned to the body through blood outlet port, 4.

FIG. 2 shows one possible implementation of the dialyzer, where blood is removed from the body through access needle 9, and returned to the body through access needle 8. Both of these can be isolated using the 3 way priming valve 7. In a preferred embodiment, the total blood required to prime the circuit is less than 100 ml, more preferably about 50 ml. There are a number of possible implementations of the exchange portion of the dialyzer. What is required is a large area of contact between the dialysate and the blood. There are a number of possible embodiments. One of these is layered flow, cross current dialysis as shown in FIG. 3. Here the dialysate runs perpendicular to the the blood, which travels in a U shaped loop. This ensures that there is the maximum concentration gradient between chemical species in the dialysate, and species in the blood. Because concentration gradient is one of the primary drivers of diffusion, this method will speed the dialysis.

In a further embodiment, as shown in FIG. 4, the dialysate, 14, and blood 15, flow in the same direction in a channel. In this particular embodiment, the channel is micro fabricated and has a width of approximately 100 microns. The channel is etched into a glass substrate, with channel side walls, 16, and channel bottom 17 being formed by the glass. Alternatively, these could be molded from a soft polymer, such as polydimethylsiloxane (PDMS). The top, 18, is closed using a separate layer of glass, or another layer of PDMS or other polymer, including heparanized polymers and more blood compatible polymers such as polyurethane.

In the preferred embodiment shown in FIG. 5, the blood, 19, and dialysate, 20, flow in a single cylindrical stream tube. The flow of blood is driven by the negative pressure gradient established by the Venturi effect as the dialysate accelerates in the tube when it moves from diameter 21 to diameter 22. Blood enters the channel at 23, through a port, and exits the channel at 24 through a second port. The distance between 23 and 24, and the flow velocity, determines the effect of the dialysis.

### BRIEF DESCRIPTION OF THE DRAWINGS

FIG. 1 is a schematic drawing of a dialyzer cartridge showing that it can be worn next to the patient's body.

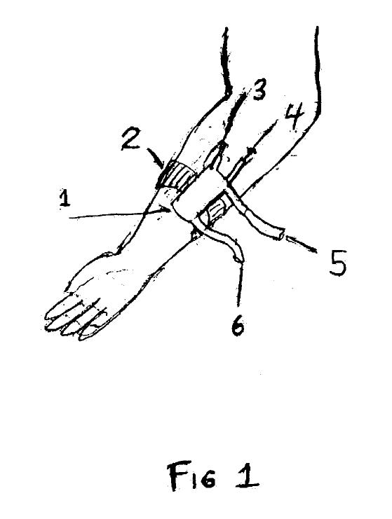

FIG. 2 is a schematic drawing of the dialyzer cartridge showing blood and dialysate inlet and outlet paths.

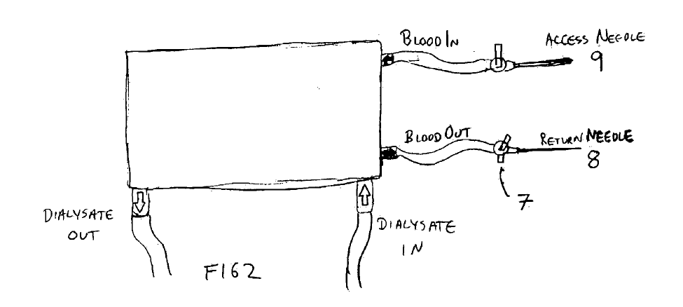

FIG. 3 is a schematic drawing showing one embodiment of the invention as a layered cross-flow dialyzer.

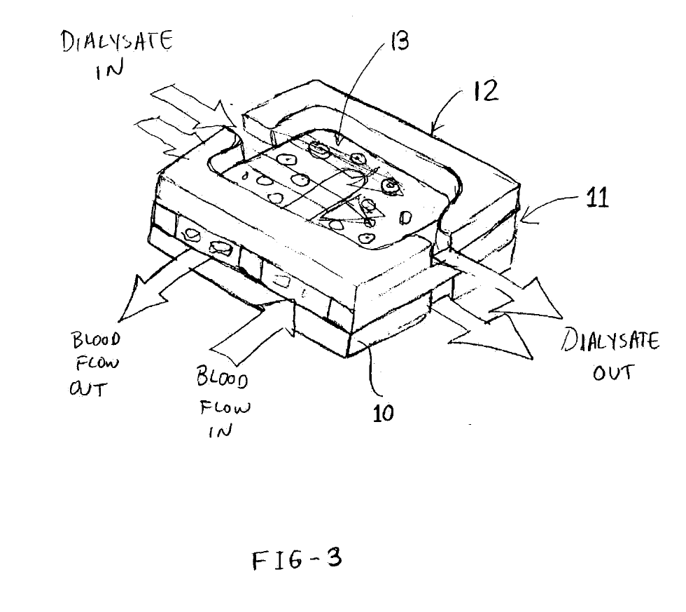

FIG. 4 is a schematic drawing of one channel of the dialyzer, showing blood and dialysate inlet and outlet structures.

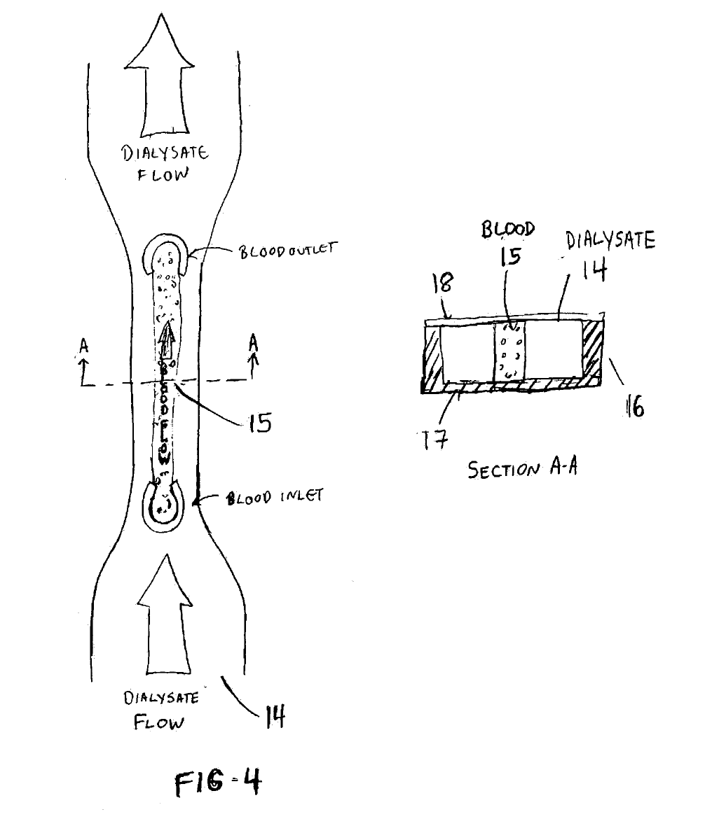

FIG. 5 is a preferred embodiment of one flow channel of the dialyzer, showing blood circumferentially surrounded by dialysate.

FIG. 6 is a detail view of a single circumferential channel showing dimensions of interest.

FIG. 7 is an arrangement of a multichannel dialyzer cartridge that can be fabricated by assembling multiple thin laminate layers.

FIG. 8 is an alternative fabrication arrangement showing multiple channels fabricated in a thin layer and then assembled by rolling the layer.

FIG. 9 is a detailed view of the texturing field that could be applied to the inside of a channel in order to promote the formation of a pseudo-intima.

FIG. 10 is a detail view showing a fluid focusing mechanism that can be used to achieve volume control.

FIG. 11 is a schematic drawing of a portable dialysis system being worn by a patient, including reservoir, dialysis cartridge and waste reservoir.

FIG. 12 is a schematic drawing showing one embodiment of the filter using micro-needles to inject the blood into a micro-machined carrier for the dialysate.

### Detailed Description of the Invention.

There are two primary functions of the kidneys—the removal of toxins, and the control of blood volume through the elimination of excess water. Thus, it must be the aim of any hemodialysis system to both filter toxins and concentrate the blood. In current systems, filtration takes place using a semi-permeable membrane to prevent large molecules and cells from passing from the blood into the dialysate. The flow of toxins from the blood to the dialysate is governed by the concentration difference between the blood and the dialysate, the pressure applied to the blood, and the permeability of the membrane to the particular toxin.

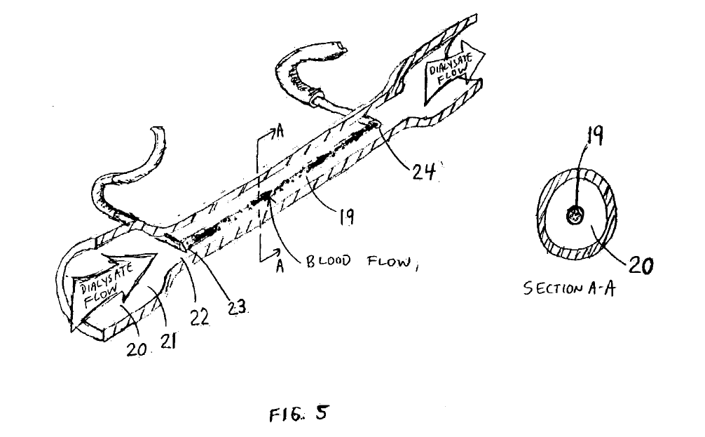

In the current invention, there is no membrane that separates the two fluids from each other. Instead the flows are kept in intimate contact with each other. The transport of matter between the flows can be separated into two components—convective and diffusive transport. Convective transport is dominant in flows where inertial forces are a primary force, whereas diffusion is dominant in the situation where viscous forces are dominant. These two regimes can be understood by examining the Reynolds (Re) number for the flow, which is the ratio of inertial to viscous forces. If this number is low enough, the flows are said to be reversible and transport between the two will be dominated by diffusion, rather than convection. Empirical experimental evidence shows that for flow in pipes, keeping the Re<2300 will keep the flows laminar and dominated by viscous forces. Flows of this nature are said to be reversible and the only motion of constituents of the flow from one streamline to another will be driven by diffusion. In order to design a hemodialysis filter based on this phenomenon, we need to determine equations that allow us to determine the rate of removal of contaminants from the blood and the pressure drop through a particular device. FIG. 5 shows one possible implementation of the hemodialysis filter, where the blood 19, and dialysate, 20, flow in the same direction. While these equations are being developed for this example it should be appreciated that they can be readily extended to other configurations by one reasonably skilled in the art.

We assume that the device will require more than one channel to perform dialysis in reasonable time. We also assume that it is desired to remove 95% of the contaminants in the blood in one pass through the dialyzer. This can be most easily considered as a dilution problem, and if the dialysate and blood are kept in a ratio of 20:1, the final concentration of contaminant in the aggregate solution will be 5%. Therefore we define,

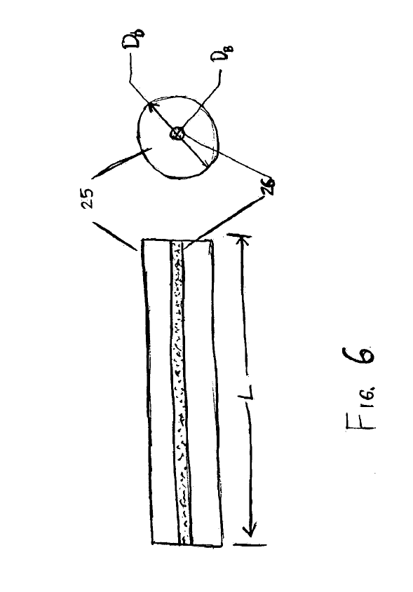

V_{dialysate}=20·V_{blood} (1)

where V_{dialysate} is the volume of dialysate supplied, and V_{blood} is the volume of blood supplied. Now recognize that in the device of FIG. 5, the blood is a cylinder entirely contained within a cylinder of dialysate with dimensions defined in FIG. 6. Therefore

$${V}_{dialysate} = \frac{π}{4} \cdot {D}_{D}^{2} \cdot L \qquad ( 2 )$$

where D_{D} is the diameter of the flow tube of dialysate, L is the length of the flow tube and

$${V}_{blood} = \frac{π}{4} \cdot {D}_{B}^{2} \cdot L \qquad ( 3 )$$

where D_{B} is the diameter of the blood tube contained within the dialysate tube. Substituting 2 and 3 into 1, and simplifying we arrive at the first design equation,

D_{D}=4.5·D_{B} (DE1)

The basic principal of operation of this invention is that molecules of different sizes diffuse at different rates. The process of diffusion is governed by Fick's law, which can be written for a one dimensional system as,

$$J = - D \frac{ⅆ n}{ⅆ x} \qquad ( 4 )$$

where D is the diffusion coefficient for a particular species, J is the diffusive flux past a particular point and dn/dx is the concentration gradient along the direction of the flux. For a sufficiently large (semi-infinite) tube, with an initial bolus of substance injected into it equation 4 has a relatively straightforward solution,

$$n ( x , t ) = {C}_{1} \cdot {ⅇ}^{( \frac{- {x}^{2}}{4 Dt} )} \qquad ( 5 )$$

where C1 is a constant related to the initial concentration of the bolus and the geometry. While this solution is by no means exact for the situation we are considering, it does give important insight into the diffusion problem and can be used to define a design equation for the system. If we consider the denominator of the exponent, we see that it has dimensions of length which is the characteristic length of the system,

L_{d}=\sqrt{Dt} (6)

Rearranging equation 6, we see that the amount of time it takes an average particle to diffuse a length Ld is related is just,

$$t = \frac{{L}_{d}^{2}}{D} \qquad ( 7 )$$

Now, if we assume that a particle has been cleared from the blood when it travels a distance L_{d}=D_{D}/4, which is a point midway between the center of the blood flow stream and the wall of the tube then equation 7 becomes our second design equation,

$$t = \frac{{D}_{D}^{2}}{16 D} . \qquad ( DE2 )$$

The diffusion of molecules can be estimated assuming they behave as small hard spheres of radius R from Einstein's equation

$$D = \frac{{k}_{b} T}{6 π μ R} \qquad ( 8 )$$

where T is the absolute temperature, k_{b} is Boltzmann's constant and μ is the viscosity of the fluid in which they are suspended. Table 1 shows the results of this calculation for some common blood components and molecular species suspended in water, at body temperature.

| | | Time to diffuse |
|---|---|---|
| Substance | Diffusivity (10⁻⁹m²/sec) | 50 μm (sec) |
| Urea | 1.67 | 0.37 |
| Glucose | 0.94 | 0.66 |
| Bilirubin | 0.718791304 | 0.87 |
| Middle Molecule (10000 | 0.138924426 | 4.50 |
| Da) | | |
| Hemoglobin | 0.1 | 6.25 |
| 0.5 μm sphere | 1.89996E−03 | 328 |
| Erythrocyte | 3.32618E−04 | 1880 |
| Phagocyte | 9.8423E−05 | 6350 |

Illustrative diffusion coefficients and diffusion times for some common blood components.

We see that there is a large separation between small molecules that need to be removed from the blood (like urea) and large molecules like hemoglobin that should remain in the blood. Thus, separation is reduced to picking a time of contact between dialysis and dialysate. In a preferred embodiment, we would like to achieve adequate clearance of molecules up to 10000 Da molecular weight. Therefore, in this embodiment, we choose to bring dialysate and blood in contact for 4.50 seconds. In order to produce a properly functioning device, we must be sure that the flows within it are always purely laminar, which implies that it has a sufficiently low ratio of inertial to viscous forces. This ratio is defined as the Reynolds number, and for flows in a tube can be written as,

$${Re}_{D} = \frac{ρ {u}_{m} {D}_{D}}{μ} \qquad ( 9 )$$

where ρ is the density of the fluid, μₘ is the mean velocity, D is the diameter of the tube and μ is the viscosity. This equation is just the ratio of inertial to viscous forces in the flow. In order to be sure that the flow is dominated by viscous forces, it is necessary to be sure that the Re number remains below 1000. We will be operating at body temperature, and can assume that in the worst case dialysate has the slightly more viscosity of water, we assume that μ=1000×10⁻⁶ Ns/m² and that ρ=1000 kg/m³. Substituting and rearranging, we arrive at design equation 3,

$${u}_{m} \leq \frac{1000 \cdot {10}^{- 6}}{{D}_{D}} \qquad ( DE3 )$$

A further constraint that can be imposed is that the pressure drop through the device needs to be kept within the physiological range. Because typical blood pressure is 130 mm/Hg systolic, and 70 mm/Hg diastolic, this implies that the pressure drop through the device can be no more than 50 mm/Hg or 6.67 kPa to allow some margin of safety. Pressure drop for laminar flow in a tube is given by the equation,

$$\frac{2 \cdot {D}_{D} \cdot Δ p}{ρ \cdot {u}_{m}^{2} \cdot L} = \frac{64}{{Re}_{D}} \qquad ( 10 )$$

and

$$\frac{L}{{u}_{m}} = 4.5$$

because the flows must remain in contact for 4.5 seconds. Now, to make sure the flow is laminar, as we have said above, Re<=1000. Substituting, rearranging and rewriting equation 10, we see that,

$${u}_{m}^{3} \leq \frac{1000 \cdot {D}_{D} \cdot Δ p}{144 \cdot ρ} \qquad ( 11 )$$

Substituting from equation DE1 we arrive at

$${u}_{m}^{3} \leq \frac{6.94 \cdot {D}_{ B} \cdot Δ p}{ρ} \qquad ( DE4 )$$

Finally, in order to achieve rapid, highly effective dialysis, we must dialyze approximately 120 L (equivalent to clearing all fluids in the body) of blood in 1 hour, or 2000 ml/min. One channel is not sufficient for this purpose, and instead we use an array of channels closely packed. The total volumetric flow rate of blood through these channels is just,

Q=0.25·n·D_{B}²·uₘ (DE5)

One preferred solution to these equations is to use an array of n=15,300 channels with D_{B}=100 μm, D_{D}=450 μm with an overall tube length of 26.7 cm. This is just one possible solution to the set of design equations, and that many more combinations are possible. This type of dialysis without a membrane is far more efficient than membrane dialysis because the solutes do not need to pass through the membrane to reach the dialysate solution. In fact, the above design solution makes possible a dialyzer that reduces the dialysis time from the current 3 to 6 hours to less than 1 hour, with equivalent clearance.

One skilled in the art will readily appreciate that because diffusion is driven by concentration differences, it is also possible to vary the concentration of a molecular species in the dialysate solution to modify the diffusion rate. This is important because one of the morbidities associated with dialysis is due to the rapid removal of salts from the blood, which forces cells to undergo rapid equilibration of intracellular and extracellular concentrations. In a rapid dialysis system as described here, this might be especially problematic as clearance of small molecules like potassium or sodium chloride, as it is likely that these would be cleared in the first few minutes of the treatmen. One possibility is to reduce the rate of clearance of these molecules by buffering the dialysate solution to control the diffusion rate with the like substance. An initially high concentration of solute in the dialysate would greatly reduce the clearance rate, which could be tapered as the dialysis procedure went on to provide effective clearance at the end of the procedure. It is also possible to use the dialysate to add components to the blood during the course of the dialysis. For example, heparin might be loaded into the dialysate, which would move into the blood and help prevent coagulation. Near the end of the procedure, the heparin could be reversed with protamine to restore normal coagulation ability. This invention contemplates a number of other possible agents that can be delivered in this manner including vitamins, pharmaceuticals or engineered hormones like erythropoetin or other red blood cell growth stimulating agents that can be used to replace the glandular function of the kidneys.

In addition to more rapid clearance, because of the nearly zero interface velocity between the blood and the dialysate, the probability of shear induced hemolysis is greatly reduced. Further, because the pressure drops through the system are kept within the physiological range (less than 50 mm/Hg) it is possible to use the dialyzer without a blood pump, which greatly reduces the complexity of the system, and allows for possible implantation of the device to be used as a true artificial kidney.

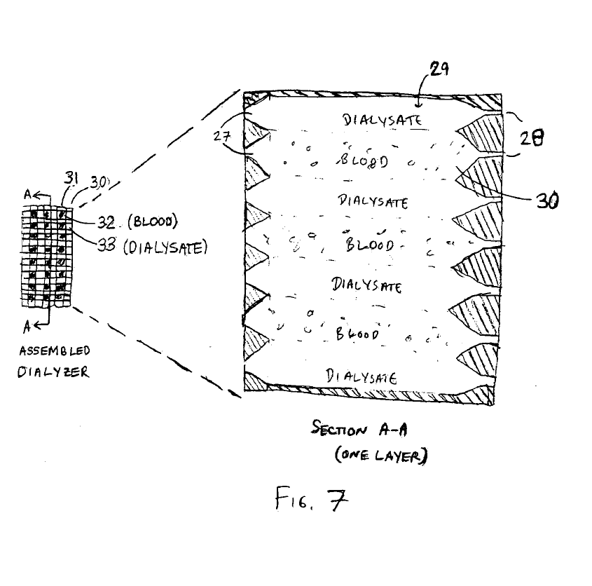

The reader should note that there are a number of other possible configurations contemplated by this invention and that this analysis is readily extendible to these different configurations. For example, FIG. 7 shows one layer of a packed array of nozzles 27, that can be used to establish an equivalent flow arrangement. Here, the dialysate, 29, and the blood, 30, move side by side through the dialyzer. By staggering the layers, 30 and 31, so that the blood channels, 32, are surrounded by dialysate channels, 33, it is possible to ensure that the blood is circumferentially surrounded by the dialysate, thus reducing contact between the blood and other foreign materials.

In the invention of FIG. 7, the ratio of dialysate to blood can be varied by varying the size of the nozzles with respect to each other, thereby changing the size of their respective flow streams. Further, in addition to diffusive clearance, convective clearance can be contemplated because the velocity of the blood can be made different than the velocity of the dialysate. This will result in a circulation from one flow to another, which is analogous to hemodialfiltration. A further embodiment of this packed array of nozzles is to alternate the hydrophobicity of the channels, 29 and 30, which will allow a direct gas to liquid interface to be developed with no infilitration of the liquid into the gas channels. One possible use of this device would be for blood oxygenation and transfer of anesthetics in cardiopulmonary bypass. It would also be possible to use a hyperoxygenated fluid in place of the dialysate, for example a fluorocarbon like synthetic cerebrospinal fluid, to provide oxygenation and carbon dioxide removal. The direct liquid to gas exchange embodiment is also useful as an artificial lung because it can be adapted to be used at normal blood pressures and normal respiration pressures and could be made from biocompatible materials that allow it to be implanted for long term use.

One method for creating alternating regions of hydrophobicity and hydrophilicity is to use a substantial hydrophobic material, such as polydimethylsiloxane (PDMS) to create the channels. Selectively masking these channels, and then exposing them to an oxygen plasma will result in the regions so exposed becoming hydrophilic, while preserving the hydrophobicity of the remainder. Similarly, chemical treatments can be contemplated, for example selectively silanizing the surface of the PDMS.

There are a number of other possible configurations for channels that have been contemplated in this invention. These include configurations where the dialysate and blood flow in opposite directions; configurations where the dialysate and blood flow at substantially right angles to each other; or other configurations where the blood flows at oblique or acute angles to the flow of the dialysate. These configurations allow the amount of convective transport between the blood and dialysate flow streams to be altered by varying the velocity difference between them. This also has the effect of altering the amount of dialysate that the blood is exposed to, which can achieve more effective dialysis in a shorter exposure time because the concentration gradient between dialysate and blood will be greater as more new dialysate flows over the blood.

There are a number of materials that the aforementioned designs can be fabricated from which include traditional microfabrication materials such as glass, fused silica, and various polymers including polydimethylsiloxane (PDMS), polymethylmethacrylate (PMMA), polycarbonate, and polytetrafluorethylene (PTFE or Teflon™). They can also be fabricated from more traditional dialyzer materials including cellulose, modified celluloses like Cuprophan™, polyurethanes and polysulfones. Each of these materials is compatible with different manufacturing methods. It is even possible to fabricate the devices from biocompatible metals like stainless steel, titanium, nickel-titanium, gold, tantalum or other biocompatible metals possessing low thrombogenicity and reactivity.

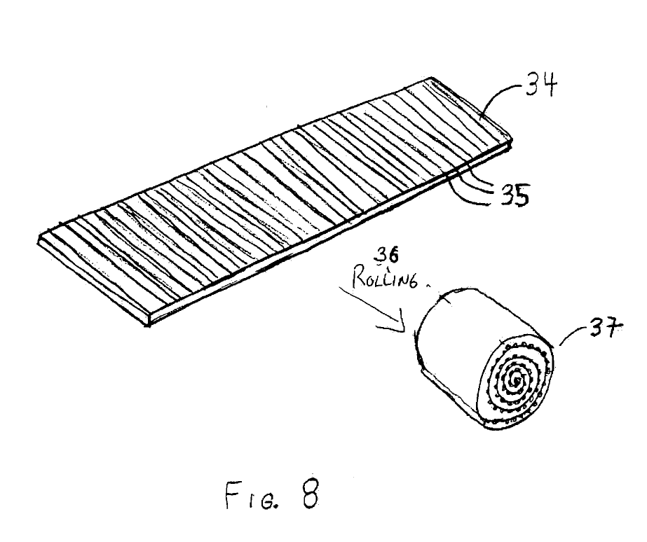

These manufacturing methods include traditional fabrication processes like extrusion as well as micro fabrication processes including hot embossing, stamping, soft lithography, micro-injection molding, sputtering and the various etching and lithographic techniques pioneered in the semiconductor industry and now used by MEMS manufacturers. FIG. 8 shows one method that is contemplated to construct a multi-layer device. In this case, a set of parallel flow channels, 35, is stamped, embossed, molded, etched or otherwise imparted onto a flat sheet, 34, of flexible material, and then this flat sheet is rolled, 36, to close the channels and construct the dialyzer, 37. Alternatively, the sheet could be folded or multiple sheets could be stacked onto each other in order to close the channels and produce the dialyzer. These sheets could be adhered together with adhesives like optically cured epoxies, cyanoacrylates, pressure sensitive adhesives or the like. They could also be thermally bonded together, or even anodically bonded.

A further objective of this invention is to reduce or eliminate the amount of anti-coagulant that is required during treatment because its use has been associated with serious morbidities and higher rates of mortality for the patient. To that end, the blood exposed surfaces of the device, including any necessary feed tubes, manifolds or cannulae that connect the dialyzer to the patient, can be pretreated with a variety of anti-thrombogenic compounds that are either coated onto or impregnated into the surface. These anti-coagulation agents include heparin, citric acid, and other compounds that interfere with the clotting cascade.

A further problem with current dialyzers is so-called “first use syndrome” where the patient experiences an adverse reaction to the dialyzer that is thought to be the result of complement activation because of exposure to fresh, somewhat biologically incompatible surfaces. One solution to this problem contemplated by this invention is to increase the biocompatibility of the device even further by pre-treating it with bovine serum albumin (BSA) or human serum albumin (HSA) or other proteins to pre-coat the surfaces with a layer of protein that reduces complement activation and prevents protein adsorption from the blood. Further modifications to the surface to increase biocompatibility or modify other surface parameters including the surface charge by coating with a different polymer, such as linear polyacrilimide, are possible.

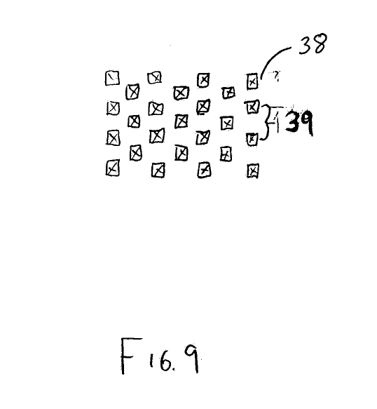

Another possible way to mitigate the patient's reaction to these filters is to texture the surface of the channels, particularly in the inlet manifold where blood is moving more slowly, to allow the development of a pseudo-intima from coagulating proteins. The texturing would encourage the formation of this intima because it would provide a nucleation site (or set of sites) for the proteins to attach themselves to. The device would be preclotted with the patients blood to produce the pseudo-intima prior to treatment. One simple pattern is illustrated in FIG. 9. This pattern is an array of pits, 38, that are most preferably 1 μm squares that are 200 nm deep. However, it is contemplated that these pits could be between 100 nm and 10 um in width, and of any possible shape including round, triangular etc. Further, these pits could be between 10 nm and 50 μm deep. In the preferred configuration, they should be spaced 2 μm on center, but can be spaced anywhere from 1.5 times their width on center to 100 times their width on center.

In another embodiment, the walls of the channels could be coated with a substance that absorbs selected contaminants in the blood. These coatings could also be design to encourage adsorption of various middle molecules in the blood, as a way of increasing the clearance rate. These coatings could also be used to promote the adherence of proteins to the channel walls to speed the process of protein coating and improve the biocompatibility further.

One of the further goals of this invention is to reduce the potential for hypovolemia of the patient by minimizing the volume of the dialyzer. This is particularly a problem with current hemodiafiltration systems, whose use often results in hypovolemia and subsequent patient morbidity. If the total blood volume of the dialyzer circuit can be kept to less than about 200 ml, the chances of producing volume depletion induced hypotension, and consequent vasoconstriction in the patient are greatly reduced. Therefore, it is a further aim of this invention to maintain dialyzer volume below 200 ml. Preventing vasoconstriction leads to improved dialysis because the capillary beds remain open, promoting scavenging of the wastes from the patient's tissues.

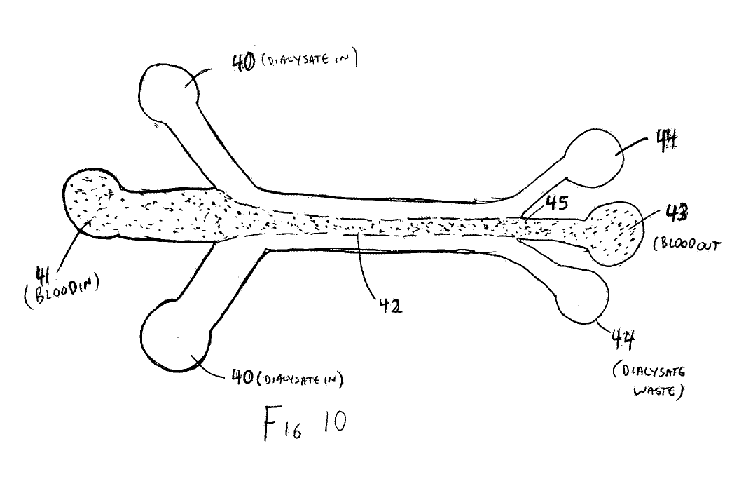

In addition to removing contaminants from the blood, an essential function of a dialysis system must be to remove excess fluid from the patients body that accumulates between dialysis treatments. As has been mentioned previously, one possible way to vary the blood volume is to vary the convective transport of fluids from blood into the dialysate by varying the velocity difference between the flow streams. A further method for achieving volume control is illustrated in FIG. 10.

FIG. 10 shows one possible channel arrangement that uses fluid focusing to achieve volume control. Focusing is achieved by varying the pressure difference between the dialysis streams, 40, and the blood stream, 41. The flow streams will focus the size of the blood stream, and force cells like erythrocytes and phagocytes to the center of the stream. These will be captured in the outlet orifice, 45, while some of the water in the blood will be diverted to the waste outlets, 44. It should be noted that for sufficiently high pressure differences between dialysate and blood, for a fixed outlet orifice, 45, blood dilution can be achieved because dialysate will be captured in the blood outlet port as well. Similarly, for sufficiently low pressure differences, it is possible to scavenge blood cells from the blood. This might be particularly useful for attaining whole blood samples for subsequent testing. In a further embodiment, electric and magnetic fields could also be used to focus the essential components of blood (erythrocytes, phagocytes, etc.) or as a way of enhancing dialysis by promoting migration of ions from the blood into the dialysate.

A further method for achieving volume control is to use the membraneless dialysis system invented herein exclusively for contaminant clearance and to use a hemodialfiltration filter, in cascade or sequentially for volume control. This has important consequences for the patients, because it makes it practical to separate filtration and volume control. Preserving a relatively large blood volume during solute clearance is beneficial because the capillary beds in the patient will remain open, and thus better clearance of solutes from the rest of the patients tissues will be achieved. Once the body has been cleared of contaminants, the volume can then be reduced using the standard filtration system. A further extension of this invention is to purposefully induce volume overload in the patient in order to cause vasodilation as the body's response to control blood pressure. Clearance of solutes from the body's tissues would become more efficient because of the better perfusion. In yet another embodiment of the dialysis system, volume control of the patient's blood is achieved by directly removing the appropriate volume of fluid from the patient's lymphatic system. Volume control can also be achieved by dialyzing the blood against non-aqueous solutions. For example, dialyzing against an alcohol such as ethanol, or a fluorocarbon or other hygroscopic solution would allow for the reduction in water content of the blood.

The amount of dilution of the blood, and the required volume reduction required can be easily monitored using inline measurements. For example, measuring the flow rate and pressure drop between the inlet and outlet ports, 27 and 28, of FIG. 7 allows the viscosity of the blood to be directly measured from Newton's law of viscosity, with little uncertainity because the exact flow conditions are known. This in turn can be correlated to the overall dilution of the blood. Dialysis can continue until the blood reaches the appropriate concentration.

Taken together, the improvements in this invention provide the enabling technology for daily, rapid dialysis that has the potential to greatly reduce patient morbidity and mortality through better control of blood volume and blood pressure, and better removal of toxins, including the so-called middle molecules which are suspected to be a primary cause of the high morbidity rates of current dialysis. Molecules such as beta-2 microglobulin, degranulation inhibitory protein I, parathyroid hormone, retinal binding protein, factor D, leptin, neopterin, adrenmedulin, advanced glycosylation end products, advanced lypooxygenation end products, neutrophil inhibitory proteins and various cytokines that have been identified as well as other products in the molecular weight range of 5000-500,000 Daltons can be cleared from the blood using this technology. Further, because the pressures are easily kept within the physiological range, and because of the elimination of the need for local anticoagulation, it is even possible to implant a filter in the patient as an artificial kidney. The patient would then only be required to change dialysate reservoirs at predetermined intervals, and could presumably experience continuous ambulatory hemodialysis, and a marked increase in quality of life.

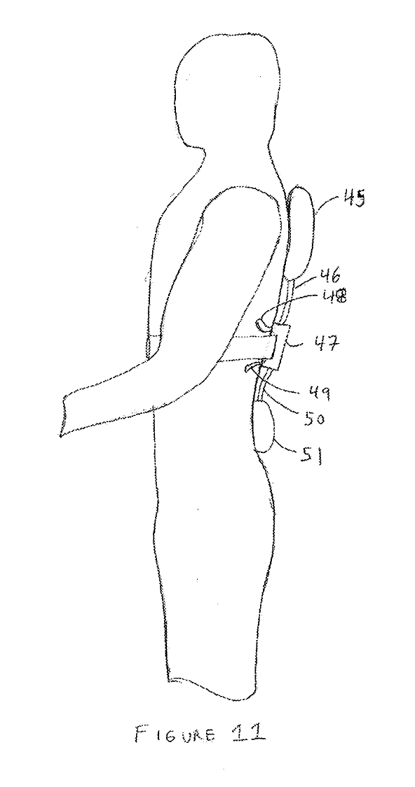

Alternatively, FIG. 11 shows a wearable, portable dialysis system that could provide continuous dialysis. The patient wears a flexible bladder in contact with the skin, 45, that serves as a dialysis reservoir and whose temperature is approximately the same as that of the patient because of the skin contact, which is connected via tubes, 46 to a dialyzer 47. The dialyzer receives blood from an arterial access 48, (implanted, for example in the hepatic artery) and returns the filtered blood through a conduit to a vein 49 (implanted, for example, in the hepatic vein, or portal vein) while dialysate waste is received in the reservoir 51. This waste can be periodically emptied by the patient and the dialysate reservoir recharged by the patient. The waste stream could also be routed to the bladder of the patient, for elimination of wastes in a more normal manner. Placing the filter and dialysate reservoir in close proximity to the body has the additional advantage of maintaining the blood and dialysate at normal body temperatures.

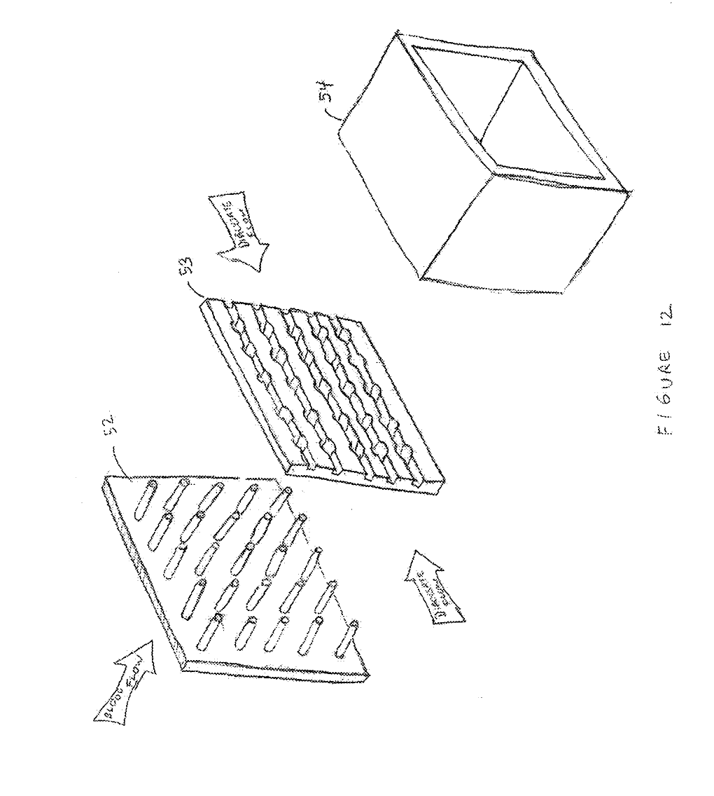

A further embodiment of the microfludic dialysis filter is shown in FIG. 12. An array of micro-needles, 52, is used to inject the blood through a dialysate manifold, 53 and into the main body of the filter, 54 where the dialysis takes place. A similar dialysate outlet manifold (not shown) and an array of outlet microneedles (also not shown) is used to separate the blood from the dialysate.

The previously discussed embodiments could be adapted to be worn as a portable or implantable artificial kidney, or could be configured to be used with a standard dialysis machine. By way of a non-limiting example, the circumferentially configured system could be built into a cartridge that would serve in place of a standard dialysis filtration unit.

The use of this invention is not limited to hemodialysis, but could enjoy a wide variety of applications. This includes the aforementioned use as a blood oxygenator. Because it can be tailored to remove larger molecules (for example by simply reducing the flow through rate for a fixed geometry) it could also be used as an artificial liver to filter toxins from the blood in emergency poisoning incidents, or as an adjuvant or replacement therapy for those with liver failure.

What is claimed is:

1. A system for performing hemodialysis on a patient including:
at least one blood channel and one dialysate channel
the at least one blood channel being circumferentially surrounded by the at least one dialysate channel
the flow in these channels being substantially inertia free
the transfer of solutes from the blood into the dialysate channel being substantially governed by the laws of diffusion.

2. The system of claim 1 where little or no blood anti-coagulation is required.

3. The system of claim 1 where the blood volume being dialyzed is substantially isovolemic or hypervolemic for the patient.

4. The system of claim 2 where volume control of the blood is achieved after solute clearance by convective clearance of liquid from the patient's blood.

5. The system of claim 2 where volume control of the patient's blood is achieved by directly removing lymph from the patient's lymphatic system.

6. The system of claim 1 where diffusion rate is modified by varying the concentration of solutes in the dialysate solution during the course of a procedure in a regulated way.

7. The system of claim 1 where the concentration of species in the dialysate is adjusted to deliver pharmaceuticals or other beneficial agents to the patient, including for example heparin and/or protamine or erythropoetin or other red blood cell production stimulating compounds.

8. The system of claim 1 where the pressure drops within the blood flow channels are kept within physiologically normal levels (<120 mm/Hg).

9. The system of claim 1 where the flow of dialysate is driven by gravity and the filter is portable and adapted to be worn externally by the patient and the pressure drops within the blood flow channels are kept to physiologically normal levels (<120 mm/Hg).

10. The system of claim 9 where a dialysate and a waste reservoir are also worn by the patient.

11. The system of claim 9 where a dialysate reservoir is worn by the patient and the waste is deposited directly into the bladder of the patient.

12. The system of claim 1 where the dialysate is a fluid whose composition has been adjusted in order to remove non water soluble compounds from the other stream.

13. A device for selectively exchanging certain components of two fluid streams that incorporates
at least one primary channel and one secondary channel
the said primary channel being at least partially surrounded by and in direct
membraneless contact with the secondary channel
the time of contact between the fluids in the two channels set at a predetermined
interval to ensure that only particular chemical species have time to diffuse from one channel to another
the flow in the primary and secondary channels being substantially unaffected by inertial effects.

14. A system for exchanging blood gases in a patient including:
at least one blood channel and one channel for gas or gas enriched fluid circulation
the at least one blood channel being at least partially surrounded by the at least one gas or gas enriched fluid channel
the flow in these channels being substantially inertia free.

15. The system of claim 20 where the channels are fabricated using alternating regions of hydrophobicity and hydrophilicity.

16. The system of claim 20 where the gas enriched fluid is an hyperoxygenated fluorocarbon, chlorofluorocarbon or synthetic cerebro-spinal fluid.

17. A system for performing substantially isovolemic or hypervolemic dialysis on a patient comprising:
at least one blood channel and one dialysate channel
the at least one blood channel being at least partially surrounded by the at least one dialysate channel and said channels maintaining pressure drops that are within physiologically normal levels (<120 mm/Hg)
the flow in these channels being substantially inertia free
the transfer of solutes from the blood into the dialysate channel being substantially governed by the laws of diffusion
the concentration of species in the dialysate is adjusted to modify the diffusion profile of species of interest
at least one connection to the patient's blood stream for removal and replacement of blood.

18. The system of claim 19 which is implanted in the patient.

19. The system of claim 1 where the dialysate is a fluid whose composition has been adjusted in order to remove non water soluble compounds from the other stream.

20. A system for performing hemodialysis containing:
at least one blood channel and one dialysate channel
the at least one blood channel being at least partially surrounded by the at least one dialysate channel and said channels maintaining pressure drops that are within physiologically normal levels (<120 mm/Hg)
the flow in these channels being substantially inertia free
the transfer of solutes from the blood into the dialysate channel being substantially governed by the laws of diffusion
the concentration of species in the dialysate is adjusted to modify the diffusion profile of species of interest
at least one connection to the patient's blood stream for removal and replacement of blood
the dialysate is a fluid whose composition has been adjusted in order to remove non water soluble compounds from the blood.
